(19) United States
(12) Patent Application Publication
(10) Pub. No.: US 2021/0337941 A1
(43) Pub. Date: Nov. 4, 2021

(54) Diffusion Apparatuses and Methods of Using the Same

(71) Applicants: Michael Nakamura, Laupahoehoe, HI (US); Jacqueline Nakamura, Laupahoehoe, HI (US)

(72) Inventors: Michael Nakamura, Laupahoehoe, HI (US); Jacqueline Nakamura, Laupahoehoe, HI (US)

(21) Appl. No.: 16/864,393

(22) Filed: May 1, 2020

(51) Int. Cl. A44C 5/00; A61L 2/26; A61L 2/20; A61Q 13/00; A61K 8/02; A01N 25/18

(52) U.S. Cl. CPC A44C 5/0084; A61L 2/26; A01N 25/18; A61Q 13/00; A61K 8/0204; A61L 2/20

(57) ABSTRACT

A diffusion apparatuses comprising a decorative element, namely a bracelet, necklace or other like decorative element that may be worn by a user thereof, diffuses substances therefrom, specifically through one or more valves. The decorative element preferably comprises a reservoir having a substance to be diffused through a tube running through the decorative element, and configured to be diffused through the one or more valves on a surface of the decorative element. Methods of using the same are further provided.

### TECHNICAL FIELD

The present invention relates to diffusion apparatuses. Specifically, the present invention comprises a decorative element, namely a bracelet, necklace or other like decorative element that may be worn by a user that diffuses substances therefrom, specifically through one or more valves. The decorative element preferably comprises a reservoir having a substance to be diffused through at least one tube running through the decorative element, and configured to be diffused through the one or more valves on a surface of the decorative element. Methods of using the same are further provided.

### BACKGROUND

Individuals often “wear” chemical substances on their bodies, clothing, or other objects for various reasons. The use of fragrances, such as perfumes or colognes, has been known for hundreds if not thousands of years. The first known chemist known to develop a perfume is believe to be a woman named Taputti who lived in Mesopotamia over 3000 years ago. Indeed, the first modern perfume was made at the behest of Queen Elizabeth of Hungary in 1370 by mixing scented oils in an alcohol solution. The use of fragrances has prospered since, and is a massive industry today.

Many airborne substances may be used for health and wellness purposes. Indeed, air sanitizers are used for disinfecting the air that people breathe. Bug repellants typically are sprayed or diffused into the air. The use of diffusers for diffusing essential oils has grown dramatically, as essential oils are used for many different purposes. For example, certain compounds may have certain physiological effects on users. Essential oils are commonly concentrated plant extracts that capture the natural smell and/or flavor, i.e., the “essence” of the plant. For example, peppermint oil may be used to boost energy and aid digestion. Lavender is commonly used to relieve stress. Sandalwood is often used to calm nerves and help with focus. Rose essential oil may be used to improve mood and reduce anxiety. Indeed, many other essential oils are known and are commonly used for a variety of reasons.

While chemicals, such as fragrances, essential oils and the like, are often used directly on skin, doing so can lead to harsh reactions. Indeed, human skin can become irritated, cracked or inflamed, react allergically, or otherwise be damaged by chemicals. Cracked skin can exacerbate the absorption of irritating chemicals, thereby causing further damage. Therefore, it is often not recommended to utilize certain chemicals directly on the skin.

Diffusion techniques are often recommended rather than placing chemicals directly on skin. Diffusion apparatuses typically intermingle volatile chemical substances with air molecules to produce airborne chemical substances. The airborne chemical substances may then be used in various ways. Airborne antiseptic substances may be used to disinfect the air within a given space, killing pathogens. Fragrances, such as perfumes, colognes and the like may be used to create pleasing scents in the air. Oftentimes, essential oils may be inhaled and used in aromatherapy. Various chemicals may also be used to aid in warding off insects, especially stinging or biting insects. However, these chemicals are often difficult to apply and are often overwhelming in their scents. Many people do not like to spray insect repellants directly on their skin or clothes.

There are various mechanisms for diffusing substances, but typically involve the use of a mechanical or electrical element to change the pressure and/or increase temperature of the substance diffused. For example, ultrasonic devices use sonic waves to cause volatile chemicals to diffuse. Electricity can be used to heat volatile chemicals causing them to diffuse into the air. And motors can be used to pressurize to push volatile substances into the air or de-pressurize to aid in volatilizing the substances. Of course, combinations of these mechanisms can also be utilized.

It is often difficult, however, to utilize diffusers in a mobile society. Oftentimes, diffusers are stationary units that do not and cannot travel with users. One way to mobilize diffusion of substances is to simply spray them onto a user or onto an object that is then carried. For example, it is common to spray fragrances, such as perfumes and colognes onto a user, or onto an object, and the fragrance diffuses from the surface of the user or object over time. However, fragrances can often lose their diffusion capability over time, often leading to a reduction in fragrance as the chemical diffuses. Moreover, once sprayed, there is no manner to increase or decrease the amount of diffusion over time. A need, therefore, exists for an improved diffusion apparatus. Specifically, a need exists for an improved diffusion apparatus that is mobile and can travel with a user. More, specifically, a need exists for an improved diffusion apparatus that maintains an amount of diffusion of a volatile substance over a period of time. Likewise, a need exists for an improved diffusion apparatus that allows a user to change the amount of diffusion at any given time, as desired by the user.

As noted, diffusers are often hard to carry with users because they are often large apparatuses, or must remain plugged into a power source to operate. A need exists for an improved diffusion apparatus that is relatively small and easily carried. Moreover, a need exists for an improved diffusion apparatus that may be operated without power, or may be operated with small and easily carried batteries.

Moreover, diffusers are often bulky and unsightly, and easily identifiable and noticeable to users. A need, therefore, exists for an improved diffusion apparatus that is relatively hidden and unnoticeable by others. More specifically, a need exists for an improved diffusion apparatus that may be shaped and sized so as to appear as other common objects that are routinely carried by users, for example as a decorative object, such as a piece of jewelry, namely a necklace or a bracelet.

### SUMMARY OF THE INVENTION

The present invention relates to diffusion apparatuses. Specifically, the present invention comprises a decorative element, namely a bracelet, necklace or other like decorative element that may be worn by a user that diffuses substances therefrom, specifically through one or more valves. The decorative element preferably comprises a reservoir having a substance to be diffused through at least one tube running through the decorative element, and configured to be diffused through the one or more valves on a surface of the decorative element. Methods of using the same are further provided.

To this end, in an embodiment of the present invention, a diffusion apparatus is provided. The diffusion apparatus comprises: a tube comprising a first end and a second end, having an outer surface and an inner surface, the inner surface forming an inner passage within the tube; at least one diffusion opening within the surface of the tube, wherein the diffusion opening forms a passage between the inner passage and the outer surface of the tube; a connector configured to connect the first end of the tube and the second end of the tube together; and a reservoir on a second end of the tube, the reservoir configured to hold an amount of a fluid substance therein, wherein the reservoir is configured to pass the fluidic substance therein to the inner passage of the tube.

In an embodiment, the diffusion apparatus further comprises a battery disposed on the first end of the tube.

In an embodiment, the battery is disposed within a battery compartment on the first end of the tube.

In an embodiment, the battery is rechargeable.

In an embodiment, the battery is rechargeable, and further comprises: a recharging port in the battery compartment configured to recharge the battery through a recharging cord.

In an embodiment, the diffusion apparatus further comprises: a motor adjacent the reservoir, wherein the motor is configured to change the pressure within the reservoir.

In an embodiment, the motor is configured to pump the fluidic substance from the reservoir into the inner passage of the tube.

In an embodiment, the diffusion apparatus further comprises: a heating element associated with the reservoir, wherein the heating element is configured to heat the fluidic substance within the reservoir.

In an embodiment, the heating element is disposed within the reservoir.

In an embodiment, the heating element is disposed around the reservoir.

In an embodiment, the diffusion apparatus further comprises: a heating element running through the tube and configured to heat the fluidic substance within the inner passage of the tube.

In an embodiment, the connector is configured to complete an electrical circuit from the first end of the tube to the second end of the tube when the connector connects the first end of the tube to the second end of the tube.

In an embodiment, the diffusion apparatus further comprises: a battery disposed on the first end of the tube, wherein the electrical circuit runs from the battery to the reservoir.

In an embodiment, the diffusion apparatus further comprises: a motor disposed on the second end of the tube adjacent the reservoir, and configured to change the pressure within the reservoir, wherein the motor is powered by the battery.

In an embodiment, the diffusion apparatus further comprises: a heating element associated with the reservoir on the second end of the tube, the heating element configured to heat the fluidic substance within the reservoir, wherein the heating element is powered by the battery.

In an embodiment, the diffusion apparatus further comprises: a motor disposed on the second end of the tube adjacent the reservoir, and configured to change the pressure within the reservoir, wherein the motor is powered by the battery; and a heating element associated with the reservoir on the second end of the tube, the heating element configured to heat the fluidic substance within the reservoir, wherein the heating element is powered by the battery.

In an embodiment, the diffusion apparatus further comprises: a pressurization bulb interconnected with the reservoir configured to move the fluidic substance from the reservoir when activated.

In an embodiment, the at least one diffusion opening is a valve.

In an embodiment, the diffusion apparatus further comprises: a volatile fluidic substance disposed within the reservoir.

In an embodiment, the diffusion apparatus is a necklace.

In an embodiment, the diffusion apparatus is a bracelet.

It is, therefore, an advantage and objective of the present invention to provide an improved diffusion apparatus.

Specifically, it is an advantage and objective of the present invention to provide an improved diffusion apparatus that is mobile and can travel with a user.

More, specifically, it is an advantage and objective of the present invention to provide an improved diffusion apparatus that maintains an amount of diffusion of a volatile substance over a period of time.

Likewise, it is an advantage and objective of the present invention to provide an improved diffusion apparatus that allows a user to change the amount of diffusion at any given time, as desired by the user.

In addition, it is an advantage and objective of the present invention to provide an improved diffusion apparatus that is relatively small and easily carried.

Moreover, it is an advantage and objective of the present invention to provide an improved diffusion apparatus that may be operated without power, or may be operated with small and easily carried batteries.

Further, it is an advantage and objective of the present invention to provide an improved diffusion apparatus that is relatively hidden and unnoticeable by others.

More specifically, it is an advantage and objective of the present invention to provide an improved diffusion apparatus that may be shaped and sized so as to appear as other common objects that are routinely carried by users, for example as a decorative object, such as a piece of jewelry, namely a necklace or a bracelet.

Additional features and advantages of the present invention are described in, and will be apparent from, the detailed description of the presently preferred embodiments and from the drawings.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 illustrates a perspective view of a diffusion apparatus in an embodiment of the present invention.

FIG. 2 illustrates a diffusion apparatus utilized as a necklace in an embodiment of the present invention.

FIG. 3 illustrates a diffusion apparatus utilized as a bracelet in an embodiment of the present invention.

### DETAILED DESCRIPTION OF THE PRESENTLY PREFERRED EMBODIMENTS

The present invention relates to diffusion apparatuses. Specifically, the present invention comprises a decorative element, namely a bracelet, necklace or other like decorative element that may be worn by a user that diffuses substances therefrom, specifically through one or more valves. The decorative element preferably comprises a reservoir having a substance to be diffused through at least one tube running through the decorative element, and configured to be diffused through the one or more valves on a surface of the decorative element. Methods of using the same are further provided.

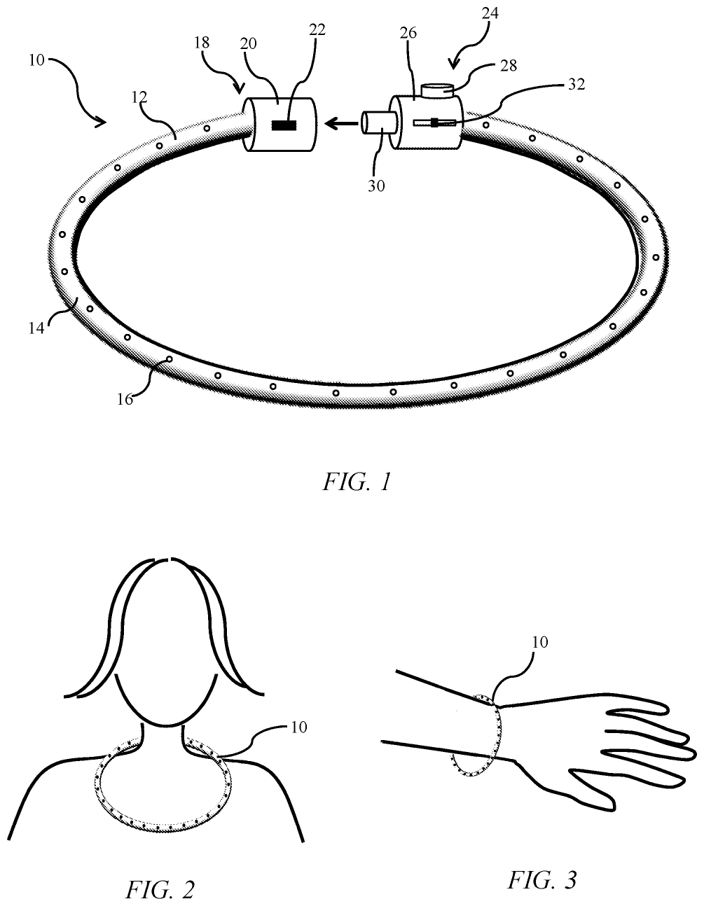

Now referring to the figures, wherein like numerals refer to like parts, FIG. 1 illustrates a diffusion apparatus 10 in preferred exemplary embodiment of the present invention. The diffusion apparatus 10 generally comprises a tube 12 generally in the shape of a hoop, although the tube 12 may be any shape and should not be limited as described herein. The tube 12 comprises an outer surface 14 and an inner surface (not shown) through which a fluidic substance may flow. Alternatively, a second tube (not shown) may be disposed within the tube 12 for distributing a fluidic substance therethrough. One or more holes or valves 16 may be disposed on the tube 12 for providing passage between the inner surface and the outer surface 14 of the tube 12, thereby allowing passage of the fluidic substance therethrough. The holes or valves 16 may be disposed in any location on the surface 14 of the tube 12 and should not be limited as described herein and shown in the figures. Specifically, a fluidic substance contained within the tube 12 may flow through the tube 12 and out the holes or valves 16 disposed within the surface 14 of the tube 12.

On a first side 18 of the tube 12 may be a battery compartment 20 comprising a battery therein, preferably a rechargeable battery that may be charged via a charging port 22, such as via a USB charger. Disposed on a second side 24 of the tube 12 may be a fluidic substance compartment 26 that may provide a reservoir for holding a fluidic substance therein. The fluidic substance may be any volatile chemical or chemical composition or mixture that may be capable of flowing through the tube 12 and through the holes or valves 16 disposed on a surface 14 of the tube 12. The fluidic substance may preferably be an essential oil, an antiseptic substance, an insect repellant, or any other volatile chemical substance that may be diffused from the apparatus 10 described herein.

The fluidic substance compartment 26 may hold an amount of the fluidic substance therein. A cap or top 28 may cover a filling aperture that may be used for filling the fluidic substance into the fluidic substance compartment 26. The cap or top 28 may be a screw cap, or otherwise provide a tight seal to prevent fluidic substance from leaking therefrom when applied thereto.

As illustrated in FIG. 1, the fluidic compartment 26 may have a peg or plug 30 that may be fit within a corresponding mating aperture (not shown) in the battery compartment 20. The peg or plug 30 may be threaded, such that the peg or plug 30 is turned and tightened onto corresponding threads within the mating aperture in the battery compartment 20. Alternatively, the peg or plug 30 may be magnetic, thereby being magnetically attracted to the mating aperture in the battery compartment 20, thereby holding the fluidic substance compartment 26 adjacent the battery compartment 20. Once brought together, a circuit may be completed, allowing the battery within the battery compartment 20 to power elements within the fluidic substance compartment 26.

Specifically, heating elements (not shown) may be contained within the fluidic substance compartment 26 for heating the fluidic substance contained within the fluidic substance compartment 26 or otherwise heating or even vaporizing the fluidic substance. Alternatively, the heating elements may be disposed around the fluidic substance compartment 26 for providing heat to the fluidic substance contained therein. When heated, the fluidic substance within the fluidic substance compartment 26 may flow therefrom, and through the tube 12, and further through the holes or valves 16 within the surface 14 of the tube 12. Additionally, heating elements may be disposed throughout the tube 12, thereby providing additional heating of the fluidic substance contained therein, thereby aiding in diffusing the fluidic substance through the holes or valves 16, as desired.

It should be noted that, in a preferred embodiment, any re-energizable component may be utilized for actively inducing the diffusion of the fluidic substance therein when desired, and should not be limited as described herein. For example, alternatively, or in addition to the heating elements described above, a motor may be contained within the fluidic substance compartment 26 or therearound for changing the pressure and/or temperature of the fluidic substance contained therein, thereby moving the fluidic substance contained therein through the tube 12 and out the holes or valves 16. For example, the motor may be utilized to pump the fluidic substance from the fluidic substance compartment 26 through the tube 12 and out the holes or valves 16. The motor that may be used to pump the fluidic substance may be joined with a heating element, as described above, thereby pumping vaporized fluidic substance through the tube 12 and out the holes or valves 16, as described herein. The inner passage, inner surface, or inner tube within the tube 12 may have a size or diameter compatible to maximize the diffusion of the fluidic substance therefrom in conjunction with the heating elements and/or motor and/or other like elements contained therein.

Moreover, an ultrasonic element may be associated with the fluidic substance compartment 26 for providing ultrasonic waves to the fluidic substance contained therein. The ultrasonic waves may add energy to the fluidic substance contained therein, moving the same through the tube 12 and out the holes or valve 16.

In an alternate embodiment of the present invention, the battery may be positioned adjacent the fluidic substance compartment 26 on the second end of the tube 12, and not on the first end thereof. Thus, the battery and the fluidic substance compartment 26 may not require an electrical circuit to be produced when the first end and the second end of the tube are connected, as described above.

A dial, switch or lever 32 may be disposed on the fluidic substance compartment 26 for increasing or decreasing the amount of flow of the fluidic substance from the fluidic substance compartment 26 and through the tube 12 and holes or valves 16. The dial, switch or lever 32 may provide control by a user thereof to the amount of fluidic substance diffusing therefrom. For example, turning the dial, switch or lever 32 one way may decrease the amount of fluidic substance diffusing therefrom, whereas turning the dial switch or lever 32 in another way may increase the amount of fluidic substance diffusing therefrom.

Diffusion of the fluidic substance contained within the diffusion apparatus 10 may be manually achieved, such as by manually activating the apparatus for diffusion therefrom. Alternatively, the fluidic substance may be automatically diffused at programmable times or via automatic time intervals, as desired by a user thereof.

Thus, the diffusion apparatus 10 may utilized as a decorative element, such as a necklace (as illustrated in FIG. 2), a bracelet (as illustrated in FIG. 3), or in other ways so as to be worn by a user thereof but having a pleasing form. The diffusion apparatus 10 may be decorated with other decorative items, such as gems, jewels, or other like items to make the diffusion apparatus 10 look more like a piece of jewelry than a diffusion apparatus. It should also be noted that there may be a plurality of tubes extending from the fluidic substance compartment within the wearable decorative element. For example, a plurality of tubes may extend from a center of a brooch, a corsage or boutonniere, pendant, or other like decorative element, each of which may be configured to allow diffusion of the fluidic substance therein. Instead of a plurality of holes, there may be a single hole on an end of the tube or tubes that is configured to diffuse the fluidic substance therefrom.

In an alternative embodiment of the present invention, the diffusion apparatus 10 may be utilized without power and/or without a motor or heating elements, wherein a fluidic substance may be contained within the fluidic substance compartment 26, and may volatilize and flow through the tube 12 and out the holes or valves 16 in other ways. For example, a bulb or other means for mechanically pressurizing the fluidic substance compartment 26 may be interconnected to the fluidic substance compartment 26. Pressing or squeezing the bulb may push the fluidic substance from the fluidic substance compartment 26, thereby aiding in diffusion of the same.

In yet another alternative embodiment of the present invention, the diffusion apparatus 10 may be utilized in non-mechanical ways. For example, simply adding body heat, such as simply disposing the apparatus 10 adjacent the user's skin, may allow the volatile fluidic substance to diffuse therefrom. In another alternate embodiment, movement of the diffusion apparatus 10, such as when disposed on a user's wrist as a bracelet or the like, may cause diffusion therefrom. For example, moving the user's hand in a motion, such as in a circular motion may cause the fluidic substance to diffuse therefrom through centrifugal force.

It should be noted that various changes and modifications to the presently preferred embodiments described herein will be apparent to those skilled in the art. Such changes and modifications may be made without departing from the spirit and scope of the present invention and without diminishing its attendant advantages. Further, references throughout the specification to “the invention” are non-limiting, and it should be noted that claim limitations presented herein are not meant to describe the invention as a whole. Moreover, the invention illustratively disclosed herein suitably may be practiced in the absence of any element which is not specifically disclosed herein.

I claim:

1. A diffusion apparatus comprising:
a tube comprising a first end and a second end, having an outer surface and an inner surface, the inner surface forming an inner passage within the tube;
at least one diffusion opening within the surface of the tube, wherein the diffusion opening forms a passage between the inner passage and the outer surface of the tube;
a reservoir on a second end of the tube, the reservoir configured to hold an amount of a fluidic substance therein, wherein the reservoir is configured to pass the fluidic substance therein to the inner passage of the tube; and
a decorative element attached to the diffusion apparatus.

2. The diffusion apparatus of claim 1 further comprising:
a battery disposed on the first end of the tube.

3. The diffusion apparatus of claim 2, wherein the battery is rechargeable.

4. The diffusion apparatus of claim 2, wherein the battery is rechargeable, and further comprising a recharging port in the battery compartment configured to recharge the battery through a recharging cord.

5. The diffusion apparatus of claim 1 further comprising:
a motor adjacent the reservoir, wherein the motor is configured to change the pressure within the reservoir.

6. The diffusion apparatus of claim 5 wherein the motor is configured to pump the fluidic substance from the reservoir into the inner passage of the tube.

7. The diffusion apparatus of claim 1 further comprising:
a heating element associated with the reservoir, wherein the heating element is configured to heat the fluidic substance within the reservoir.

8. The diffusion apparatus of claim 7 wherein the heating element is disposed within the reservoir.

9. The diffusion apparatus of claim 7 wherein the heating element is disposed around the reservoir.

10. The diffusion apparatus of claim 1 further comprising:
a heating element running through the tube and configured to heat the fluidic substance within the inner passage of the tube.

11. The diffusion apparatus of claim 1 further comprising a first connector element on a first end of the tube and a mating connector element on a second end of the tube, wherein the first and second connector elements are configured to complete an electrical circuit from the first end of the tube to the second end of the tube when the first and second connector elements connect the first end of the tube to the second end of the tube.

12. The diffusion apparatus of claim 11 further comprising:
a battery disposed on the first end of the tube, wherein the electrical circuit runs from the battery to the reservoir.

13. The diffusion apparatus of claim 12 further comprising:
a motor disposed on the second end of the tube adjacent the reservoir, and configured to change the pressure within the reservoir, wherein the motor is powered by the battery.

14. The diffusion apparatus of claim 12 further comprising:
a heating element associated with the reservoir on the second end of the tube, the heating element configured to heat the fluidic substance within the reservoir, wherein the heating element is powered by the battery.

15. The diffusion apparatus of claim 12 further comprising:
a motor disposed on the second end of the tube adjacent the reservoir, and configured to change the pressure within the reservoir, wherein the motor is powered by the battery; and
a heating element associated with the reservoir on the second end of the tube, the heating element configured to heat the fluidic substance within the reservoir, wherein the heating element is powered by the battery.

16. The diffusion apparatus of claim 1 further comprising:
a pressurization bulb interconnected with the reservoir configured to move the fluidic substance from the reservoir when activated.

17. The diffusion apparatus of claim 1 wherein the at least one diffusion opening is a valve.

18. The diffusion apparatus of claim 1 further comprising:
a volatile fluidic substance disposed within the reservoir.

19. A necklace comprising the diffusion apparatus of claim 1.

20. A bracelet comprising the diffusion apparatus of claim 1.
